(19) United States
(12) Patent Application Publication
(10) Pub. No.: US 2016/0015439 A1
(43) Pub. Date: Jan. 21, 2016

(54) Bone Screw

(71) Applicant: Dietmar Wolter, Hoisdorf (DE)

(72) Inventor: Dietmar Wolter, Hoisdorf (DE)

(21) Appl. No.: 14/754,851

(22) Filed: Jun. 30, 2015

(30) Foreign Application Priority Data
Jun. 30, 2014 (EP) 14175072.9

(51) Int. Cl. A61B 17/86

(52) U.S. Cl. CPC A61B 17/866; A61B 17/8605

(57) ABSTRACT

The invention relates to a bone screw for fixing a connection support to a bone. The bone screw comprises a screw head, which has a head thread for producing a threaded connection to the connection support. The bone screw further comprises a shank, which has a bone thread for engaging in the bone substance. Between the screw head and the shank, the bone screw has a transition area. The bone screw has a coating on its surface. According to the invention, the coating has a greater thickness in the area of the shank than in the area of the screw head. The coating according to the invention leads to an improved connecting strength between screw and connection support.

### BACKGROUND

The invention relates to a bone screw for fixing a connection support to a bone. The bone screw comprises a screw head, which has a head thread for producing a threaded connection to the connection support. The bone screw moreover comprises a shank, which has a bone thread for engaging in the bone substance. The bone screw has a transition area between the screw head and the shank. The bone screw also has a hard coating on its surface.

It is known to use bone screws in order to fix a connection support, for example a bone plate, to a bone. Bone screws are used, for example, in the treatment of bone fractures. The bone screws are guided through through-holes in the bone plate and screwed into the fragments of the broken bone. The bone thread thereby engages in the bone substance, such that a firm connection is established between bone screw and bone. Moreover, the head thread establishes a firm threaded connection to the bone plate. The fracture is in this way bridged by the bone plate, such that the fragments are stabilized and can grow together again. Bone screws can also be used together with other types of connection supports, for example elements of joint prostheses.

It is also known to provide bone screws with a hard coating. Hard coatings serve to increase the mechanical load-bearing capacity and, for example, to reduce or prevent abrasion of the material of the bone screw, during the screwing-in procedure, and an associated formation of particles. Abrasion particles are undesirable, since they represent foreign bodies within the bone tissue and may lead to adverse reactions.

The layer thickness of the coating is usually dimensioned such that it prevents direct contact between the coated material of the bone screw and the surrounding substance, this despite the loads that develop as the bone screw is being screwed in. Contact between the coated material and the bone tissue can be avoided in particular in the area of the shank. This is desirable since the core material may be attacked by amino acids present in the bone substance. However, hard coatings of this kind have the disadvantage that they are not conducive to the strength of the threaded connection between the screw head and the connection support.

### SUMMARY

Proceeding from this, the object of the present invention is to make available a bone screw which has improved connecting strength between the head of the bone screw and the connection support. This object is achieved by the features of the independent claims. Advantageous embodiments are set forth in the dependent claims.

According to the invention, the coating has a lesser thickness in the area of the screw head than in the area of the shank.

First, some of the terms used in the context of the invention will be explained. A coating is designated as hard when it has a greater hardness than the coated material. In the determination of relative hardness, the different techniques used to measure hardness generally lead to identical results. For example, the Rockwell hardness can be determined (DIN EN ISO 6508-1).

A transition area of the bone screw designates a part of the screw which, in the longitudinal direction of the screw, is arranged between the screw head and the shank. In the transition area, the layer thickness of the coating is not fixed. For example, the layer thickness at the shank-side end of the transition area can correspond to the layer thickness on the shank. At the head-side end of the transition area, the layer thickness can correspond to the layer thickness on the screw head. Between the shank-side end and the head-side end, the layer thickness can vary in any desired way, for example it can decrease continuously.

The invention is based on the understanding that a hard coating with great thickness in the area of the screw head can be disadvantageous and can adversely affect the strength of the connection between the head thread and the connection support. This is especially the case when bone screw and connection support are made of the same type of material, for example of titanium alloys or of pure titanium. In this case, the hard coating in fact prevents the materials of the same type from coming into direct contact. The invention has recognized, however, that direct contact between screw head and connection support is advantageous, since the strength of the connection is increased on account of a chemical bond between the same types of materials. Since the invention now proposes that a thinner coating be provided on the screw head, it is possible to ensure, when producing the threaded connection between screw head and connection support, that a screw head and connection support are at least partially in direct contact and are able to form a firm connection to each other. Notwithstanding this, the coating is still thick enough to ensure that, in the areas adjacent to the threaded connection, it prevents free contact between the coated material and the surrounding tissue.

The head thread according to the invention for producing a threaded connection to the connection support can be designed as a thread for engagement in a mating thread of the connection support. The head thread can also be designed as a self-cutting thread which, as it is being screwed in, deforms the material of the connection support in order to produce the threaded connection.

In an advantageous embodiment, the coating in the area of the screw head has a thickness of between 10 nm and 70 nm, preferably of between 20 nm and 50 nm. A chosen coating thickness of this kind in the area of the screw head has the effect that the materials of the same type for the screw head and for the connection support come to lie directly on each other. This leads to a firm connection between screw head and connection support.

The coating in the area of the shank can have a thickness of between 80 nm and 5 μm. Production is simpler if the layer thickness on the shank is only slightly greater than on the screw head and is, for example, between 80 nm and 200 nm, preferably between 90 nm and 120 nm, more preferably about 100 nm. In order to separate the coated material reliably from the environment, a layer thickness at the upper end of the claimed range is advantageous, for example between 1 μm and 5 μm. As a result of the greater layer thickness in the area of the shank, the screw withstands the loads that arise as the screw is being screwed in. In this way, the coated material on the shank of the bone screw remains protected by the coating against direct contact with the bone substance.

In an advantageous embodiment, the coating of the bone screw comprises titanium oxide. The term titanium oxide comprises TiO₂, but also the suboxides of titanium such as Ti₂O₃ or Ti₃O₅. The coating can be produced, for example, by anodic oxidation of the bone screw. With this method, a hard coating can be generated in a simple way. The titanium oxide preferably forms the main constituent of the coating. The coating of the bone screw can also consist entirely of titanium oxide.

In a preferred embodiment, the bone screw consists of pure titanium, more preferably of pure titanium grade 4. The bone screw can also consist of a titanium alloy, preferably of TiAl6V4.

The subject matter of the present invention also concerns a fixation system for bones, with a connection support having at least one through-hole, and with at least one bone screw according to the invention. The bone screw can be inserted into the through-hole in order to produce a threaded connection between the head thread and the connection support. The connection support is preferably made of pure titanium or a titanium alloy. This means that the pure titanium or the titanium alloy forms the main constituent of the connection support. For example, the connection support, apart from the coating, can consist entirely of pure titanium or the titanium alloy. The titanium alloy can be TiAl6V4, for example.

In the area of a through-hole, the connection support can have a mating thread for the head thread of the bone screw.

In a preferred embodiment, however, the connection support is designed, in the area of a through-hole, in such a way as to be deformed by the head thread of the bone screw when a threaded connection is produced. For this purpose, in the area of a through-hole, the connection support preferably consists of a material that is softer than the material of the bone thread. In this way, the angle adopted by the bone screw relative to the connection support, after the threaded connection is produced, can be chosen freely within a range of angles. It is possible in this way to produce a multi-directional and angle-stable connection between bone screw and connection support.

In an advantageous embodiment, the connection support comprises, in the area of a through-hole, a material lip which, upon production of a threaded connection, is deformed by the head thread of the bone screw. The material lip can be formed by a peripheral projection on the inner face of a through-hole. The material lip preferably consists of a softer material than the rest of the connection support.

### BRIEF DESCRIPTION OF THE DRAWINGS

The invention is explained below with reference to the attached drawings in which an advantageous illustrative embodiment is depicted. In the drawings:

FIG. 1 shows a side view of a bone screw according to the invention;

FIG. 2 shows a partial detail of the bone screw from FIG. 1 in a sectional side view before the bone screw is screwed into a bone or a connection support;

FIG. 3 shows a partial detail of the bone screw from FIG. 1 in a sectional side view after the bone screw has been screwed into the bone or into the connection support;

FIG. 4 shows a further embodiment of a bone plate according to the invention in a sectional side view;

FIG. 5 shows a fixation system according to the invention in a sectional side view.

### DETAILED DESCRIPTION

FIG. 1 shows a side view of a bone screw. At its upper end, the bone screw has a screw head 13 and, below the screw head 13, it has a shank 14. The diameter of the screw head 13, at the lower end thereof, is substantially identical to the diameter of the shank 14. The diameter of the screw head 13 increases in the direction of the upper end. A tool seat 19 is let into the screw head 13 and allows the bone screw to be turned with the aid of a suitable tool.

A head thread 15 is arranged on the screw head 13. Moreover, a bone thread 16 is located on the shank 14. The bone screw consists of the titanium alloy TiAl6V4, the bone screw having been provided with a titanium oxide layer by anodic oxidation. The titanium oxide layer is shown in FIG. 2.

FIG. 2 shows a partial detail of the bone screw from FIG. 1 in a sectional side view. The section extends through the longitudinal axis of the bone screw. FIG. 2 shows the bone screw, and in particular the coating of the bone screw, in a state before it is screwed into a bone or into a bone plate. For purposes of illustration, the thickness of the coating is not shown true to scale but instead much exaggerated.

In the area of the head thread 15, the bone screw is provided with a titanium oxide coating 17 which has a thickness of 30 nm. In the area of the shank 14, the bone screw is provided with a titanium oxide coating 18 which has a thickness of 100 nm. On account of its greater thickness, the coating 18 is much more resistant to friction than the coating 17.

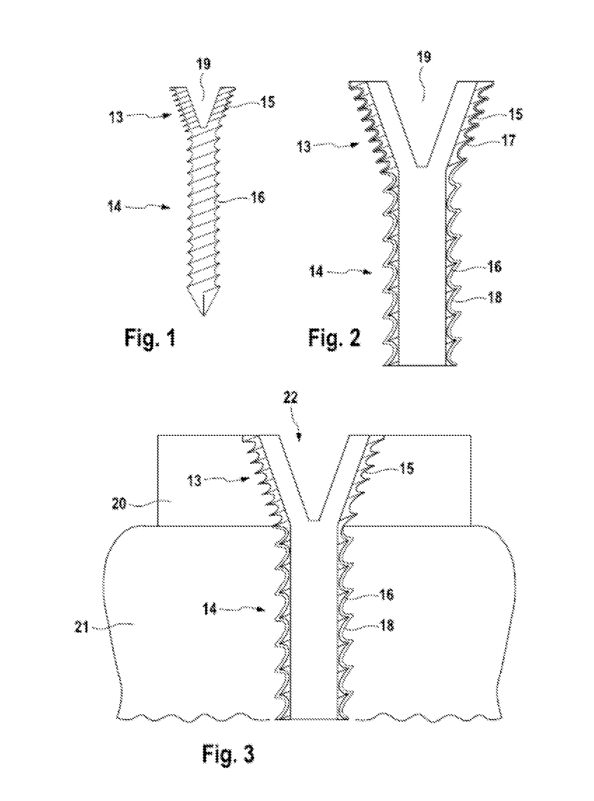

The state of the bone screw after it has been screwed into a bone 21 and into a bone plate 20 lying on the bone 21 is illustrated in FIG. 3. The bone plate 20 consists of a titanium alloy and has a through-hole 22. The bone screw was guided through the through-hole 22 and screwed into the bone 21. The bone thread 16 was thus brought into engagement with the bone substance of the bone 21, such that a firm connection arises between the shank 14 and the bone 21. Moreover, the head thread 15 was brought into engagement with the bone plate 20. Despite the strong friction between the bone substance and the shank 14, the thicker coating 18 separates the coated material of the shank 14 from the surrounding bone substance. In contrast to this, there is no complete separation in the area of the thin coating 17, such that the head thread 15 is in direct contact with the bone plate 20.

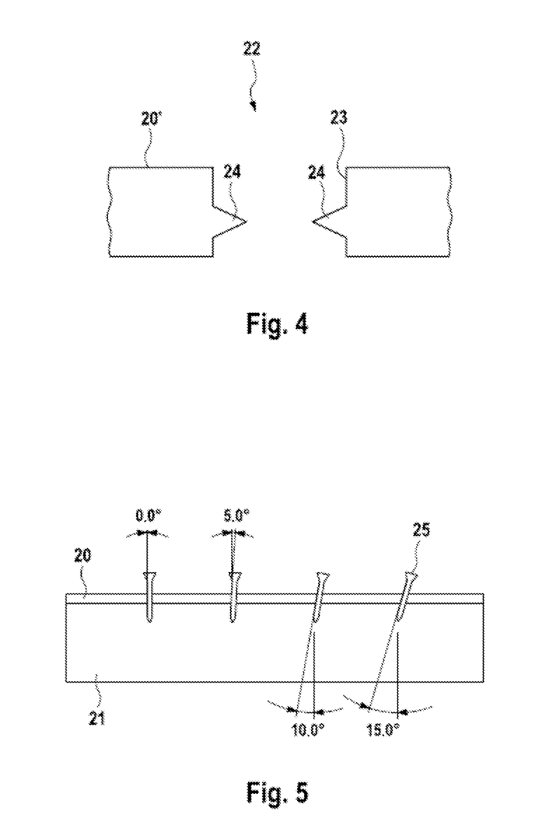

FIG. 4 shows a further embodiment of a bone plate 20′ according to the invention in a sectional side view. The bone plate 20′ has a through-hole 22, on the inner face 23 of which an inwardly protruding lip 24 is arranged all the way round. When a threaded connection is produced between the head thread of a bone screw and the bone plate 20′, the lip 24 is deformed by the head thread of the bone screw. The angle between the bone screw and the bone plate can be chosen freely here within certain limits.

FIG. 5 shows a fixation system according to the invention with a bone plate 20 and four bone screws 25 in a sectional side view. The bone plate 20 lies on a bone 21. The bone screws 25 can be screwed at freely selectable angles of between 0° and 15° into the bone plate 20 and into the bone 21. The connection is thus variable in terms of its angle. As a result of the threaded connection between the head of the bone screw 25 and the bone plate 20, the connection is also stable in terms of its angle, i.e. set at a fixed angle between the bone screw 25 and the bone plate 20.

1. A bone screw for fixing a connection support to a bone, with a screw head, which has a head thread for producing a threaded connection to the connection support, with a shank, which has a bone thread for engaging in the bone substance, and with a transition area between the screw head and the shank, the bone screw having a hard coating on its surface, wherein the coating has a lesser thickness in the area of the screw head than in the area of the shank.

2. The bone screw according to claim 1, wherein the coating in the area of the screw head has a thickness of between 10 nm and 70 nm, preferably of between 20 nm and 50 nm.

3. The bone screw according to claim 1, wherein the coating in the area of the shank has a thickness of between 80 nm and 5 μm.

4. The bone screw according to claim 1, wherein the coating comprises titanium oxide.

5. The bone screw according to claim 1, wherein the coating consists of titanium oxide.

6. The bone screw according to claim 1, wherein the bone screw consists of pure titanium, preferably of pure titanium grade 4.

7. The bone screw according to claim 1, wherein the bone screw consists of a titanium alloy, preferably of TiAl6V4.

8. A fixation system for bones, with a connection support having at least one through-hole, and with at least one bone screw according to claim 1, wherein the bone screw is inserted into the through-hole in order to produce a threaded connection between the head thread and the connection support.

9. The fixation system according to claim 8, wherein the connection support consists essentially of titanium.

10. The fixation system according to claim 8, wherein the connection support comprises, in the area of a through-hole, a material lip which, upon production of a threaded connection, is deformed by the head thread of the bone screw.
